(19) United States
(12) Patent Application Publication
(10) Pub. No.: US 2007/0060901 A1
(43) Pub. Date: Mar. 15, 2007

(54) Disposable incontinence napkin for men

(76) Inventor: George Alletsee, Brandon (CA)

Correspondence Address: LITMAN LAW OFFICES, LTD, PO BOX 15035, CRYSTAL CITY STATION, ARLINGTON, VA, 22215, US

(21) Appl. No.: 11/372,263

(22) Filed: Mar. 10, 2006

Related U.S. Application Data
(60) Provisional application No. 60/715,171, filed on Sep. 9, 2005.

(51) Int. Cl. A61F 13/15

(52) U.S. Cl. 604/385.09; 604/385.03

(57) ABSTRACT

The disposable incontinence napkin for men is an elongated sanitary napkin having a liquid permeable top layer that faces the pubic area in use, a liquid impermeable bottom layer that faces the undergarment in use, and an absorbent middle layer disposed between the top and bottom layers for absorbing urine. The sanitary napkin has an anterior portion that overlies the base of the penis and upper pubic area, a posterior portion that overlies the scrotum, and a medial portion extending between the anterior and posterior portions. A posterior shield of liquid impermeable material covers the posterior portion of the top layer, and a soft layer of cotton is disposed over the posterior shield. Adhesive strips disposed on the bottom layer adhere the sanitary napkin to the undergarment and prevent bunching of the napkin.

### CROSS-REFERENCE TO RELATED APPLICATION

This application claims the benefit of U.S. Provisional Patent Application Ser. No. 60/715,171, filed Sep. 9, 2005.

### BACKGROUND OF THE INVENTION

1. Field of the Invention

The present invention relates to incontinence napkins. More particularly, the present invention relates to a disposable incontinence napkin for men.

2. Description of the Related Art

Disposable sanitary napkins worn in the crotch area for the absorption of body exudates during menstruation by women are well known and widely used. Such napkins are also useful for the absorption of minor amounts of urine by those suffering from incontinence. The typical sanitary napkin has a liquid permeable upper layer, a liquid impermeable bottom layer, and an absorptive material encased therebetween. When urine is discharged into the napkin, the entire liquid permeable layer becomes wetted due to wicking of the material and gravity flow. When used by a male person, the scrotum comes in contact with the upper layer, which, upon wetting of the upper layer with urine, causes itching and discomfort to the user in the scrotum area. Moreover, when wet urine contacts the scrotum, it creates an extremely offensive odor.

It would be desirable to provide a disposable sanitary napkin useful by men with incontinence that avoids discomfort to the scrotum after wetting with urine. Also, present sanitary napkins tend to bunch in the area of the scrotum causing further discomfort to men. It would be desirable to provide a sanitary napkin for men that does not bunch in the area of the scrotum. Thus, a disposable incontinence napkin for men solving the aforementioned problems is desired.

### SUMMARY OF THE INVENTION

The disposable incontinence napkin for men is an elongated sanitary napkin having a liquid permeable top layer that faces the pubic area in use, a liquid impermeable bottom layer that faces the undergarment in use, and an absorbent middle layer disposed between the top and bottom layers for absorbing urine. The sanitary napkin has an anterior portion that overlies the base of the penis and upper pubic area, a posterior portion that overlies the scrotum, and a medial portion extending between the anterior and posterior portions. A posterior shield of liquid impermeable material covers the posterior portion of the top layer, and a soft layer of cotton is disposed over the posterior shield. Adhesive strips disposed on the bottom layer adhere the sanitary napkin to the undergarment and prevent bunching of the napkin.

The posterior shield provides a liquid impermeable barrier between the posterior portion of the absorbent pad and the scrotum so that the scrotum remains dry and is insulated from urine that is absorbed by the absorbent middle layer. The soft layer of cotton or other soft fabric is disposed over the shield to provide a comfortable surface adjacent the scrotum in order to prevent chaffing. The adhesive strips may be disposed along the lateral edges of the posterior portion of the bottom layer, and a wider, centrally disposed adhesive strip may extend longitudinally from the anterior edge to the posterior edge of the bottom layer.

These and other features of the present invention will become readily apparent upon further review of the following specification and drawings.

### BRIEF DESCRIPTION OF THE DRAWINGS

FIG. 1 is a top plan view of a disposable incontinence napkin for men according to the present invention.

FIG. 2 is a section view drawn along section lines 2-2 of FIG. 1.

FIG. 3 is a bottom view of the disposable incontinence napkin of FIG. 1.

FIG. 4 is a top plan view of an alternative embodiment of a disposable incontinence napkin for men according to the present invention.

Similar reference characters denote corresponding features consistently throughout the attached drawings.

### DETAILED DESCRIPTION OF THE PREFERRED EMBODIMENTS

The present invention is a disposable incontinence napkin for use by men. The incontinence napkin incorporates a sanitary napkin-having a general structure similar to a conventional disposable menstrual sanitary napkin for women. The sanitary napkin is modified by adding at least a layer of material impermeable to liquids, such as urine, to the posterior portion of the top layer of the napkin, which is disposed between the scrotum and the absorbent pad of the sanitary napkin, in order to prevent contact of the wet portion of the napkin with the scrotum of a male user.

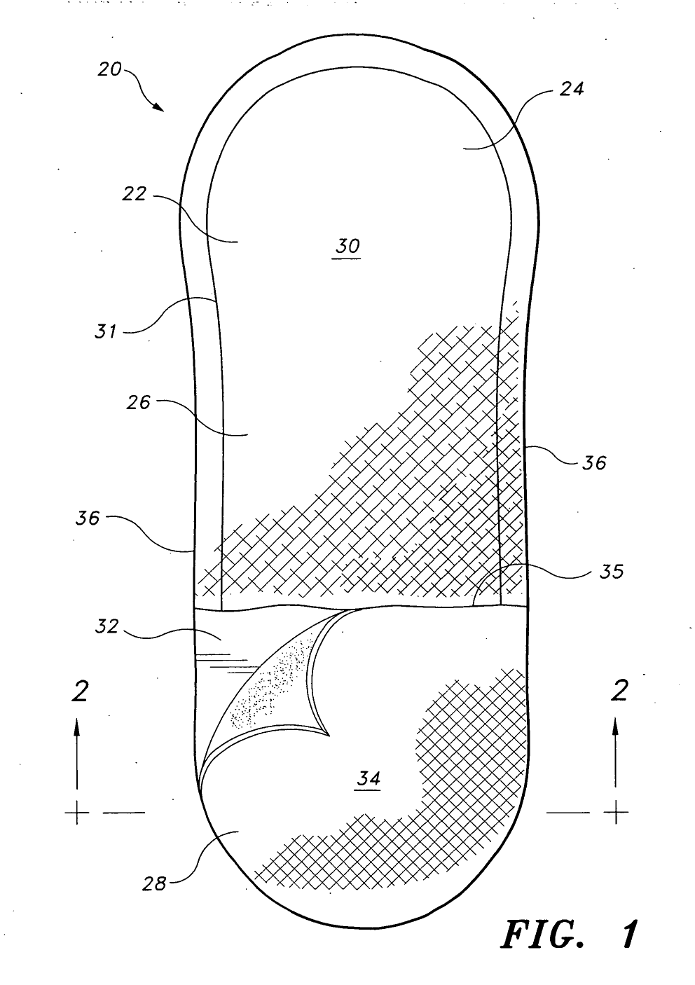

Referring to FIGS. 1-3, there is shown a plan view, a sectional view, and a bottom view, respectively, of the disposable incontinence napkin for men, designated generally as 20 in the drawings. Incontinence napkin 20 includes an elongated sanitary napkin 22 having a structure corresponding to a sanitary napkin typically used by women as a panty liner. Sanitary napkin 22 has a liquid impermeable bottom layer 40, which normally faces the undergarment, a liquid permeable top layer 30, which normally faces the pubic area, and an absorbent middle layer 44, which is disposed between the bottom layer 40 and the top layer 30. The sanitary napkin 22 may have a peripheral seam 31 joining the top layer 30 and the bottom layer 40 together and enclosing the middle layer 44 therebetween

Bottom layer 40 may be made from a plastic or flexible polymer film. Top layer 30 may be made from an absorbent web of synthetic polymeric material, and may be perforated. Top layer 30 should, however, help to wick moisture into absorbent middle layer 44 and minimize leakage back out of sanitary napkin 22. Absorbent middle layer may be made from an absorbent fluff or loose agglomeration of natural or synthetic fibers. Sanitary napkins are well known in the art, and any materials conventionally known in the art for providing a liquid permeable layer, a liquid impermeable layer, and an absorbent middle layer therebetween may be used in constructing the elongated sanitary napkin 22 of the present invention. An exemplary sanitary napkin that may be incorporated into the present invention is a Kotex “Maxi” pad or napkin made by Kimberly-Clark Corporation, Neenah, Wis. However, other similar sanitary napkins and designs may be used.

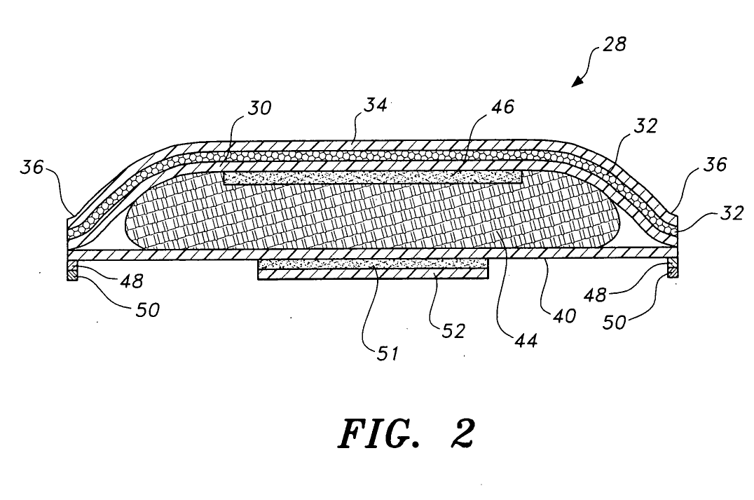

As shown in FIG. 2, the sanitary napkin 22 may also include an absorbent pad 46, e.g., a mat or web of absorbent fibers, to maximize the liquid-holding capacity of the sanitary napkin 22. The absorbent pad 46 also helps maintain the elongate shape of the sanitary napkin 22, and to assist in the distribution of liquid exudates to the absorbent middle layer 44.

The sanitary napkin 22 has an anterior portion 24 that is disposed opposite or overlies the base of the penis and upper pubic area when the incontinence napkin 20 is in use. The sanitary napkin has a posterior portion 28 that is disposed opposite or overlies the scrotum when the incontinence napkin 20 is in use. The sanitary napkin 22 has a medial portion 26 that extends between the anterior portion 24 and the posterior portion 28. The sanitary napkin 22 may have lateral recesses formed generally in the medial portion 26 for conforming to the contour of the thighs, although lateral recesses are not requisite in the present invention.

The disposable incontinence napkin 20 of the present invention supplements the sanitary napkin 22 with a posterior shield 32 made from a liquid impermeable material that covers the posterior portion 28 of the top layer 30 of the sanitary napkin 22. The posterior shield 32 may be made from a polymer film, and may be made from the same material as bottom layer 40. Posterior shield 32 prevents the possibly wet surface of top layer 30 from coming into contact with the scrotum of the user, providing a barrier against moisture. The posterior shield 32 preferably extends from the posterior edge of the sanitary napkin 22 about three inches towards the medial portion 26 of the napkin 22, the leading edge 35 of the peripheral shield 32 generally denoting the boundary between the posterior portion 28 and the medial portion 26. The posterior shield 32 covers substantially the entire surface area of the top layer 30 between the lateral edges 36 of the sanitary napkin 22, and between the posterior edge and the boundary between the posterior portion 28 and the medial portion 26 of the sanitary napkin 22, and may be adhered or otherwise bonded thereto over the entire overlapping surface area, or about the periphery of the posterior shield 32.

A layer 34 of soft fabric, such as cotton or other natural or synthetic material, is disposed over the entire surface area of the posterior shield 32. The soft fabric layer 34 provides comfort for the scrotum and prevents the posterior shield 32 from chaffing or otherwise irritating the scrotum. The soft fabric layer 34 may be adhered or bonded to the posterior shield 32 over the entire surface area of the shield 32, or only about the periphery of the shield 32.

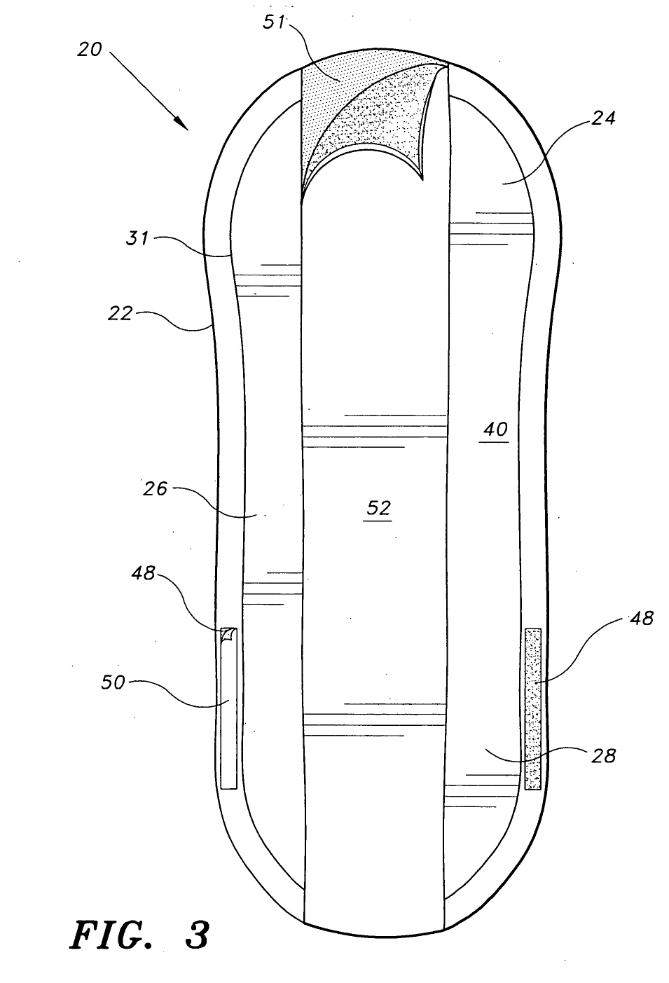

As shown most clearly in FIG. 3, adhesive strips 48 may be disposed along the lateral edges 36 of the posterior portion 28 on the bottom layer 40 of the incontinence napkin 20. The adhesive strips 48 may be covered by a peel-off paper backing 50 until the napkin 20 is ready for use. The adhesive strips 48 are used to attach the posterior portion 28 of the napkin 20 to the undergarment, thereby disposing the posterior portion opposite the scrotum and preventing the lateral edges 36 of the napkin 20 from bunching up around the scrotum. A wider, centrally disposed strip of adhesive 51 extends longitudinally on the bottom layer 40 of the napkin 20, and is covered by a peel-off paper backing 52 until the napkin 20 is ready for use. The central strip of adhesive 51 is used to adhere the incontinence napkin 20 to the undergarment throughout the length of the napkin 20, disposing the napkin opposite the pubic area.

In use, the paper backings 50 and 52 are peeled off the bottom layer 40 and the incontinence napkin 20 is attached to the inside of the undergarment. The anterior portion 24 overlies the upper pubic area and base of the penis, the posterior portion 28 overlies the scrotum, and the medial portion 26 overlies the intermediate region. When urine is excreted, the urine is wicked up by the top layer 30 and absorbed by the middle layer 44, the bottom layer 40 keeping the undergarment dry. The posterior shield 32 provides a moisture-proof barrier between the top layer 30 and the scrotum, keeping the scrotum dry. The soft fabric layer 34 cushions the scrotum and prevents the shield 32 from chaffing the scrotum. Adhesive strips 48 prevent the posterior portion from bunching up and causing discomfort to the scrotum.

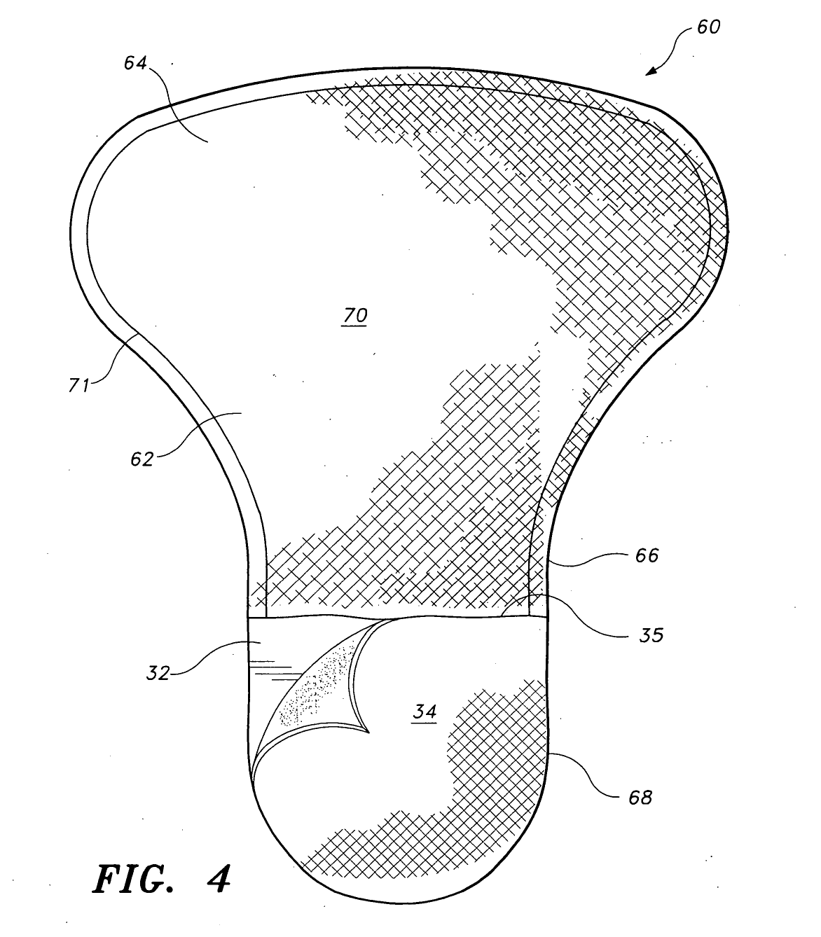

FIG. 4 shows an alternative embodiment of a disposable incontinence napkin for men, designated generally as 60 in the drawing. The construction of the incontinence napkin 60 is substantially identical to the first embodiment of the napkin 20, except that the anterior portion 64 of the sanitary napkin 62 is substantially wider than the anterior portion 24 of sanitary napkin 20 in order to overlie a larger portion of the upper pubic area. The medial portion 66 tapers in width from the anterior portion 64 to the posterior portion 68. The top layer 70 is bonded to the bottom layer around peripheral seam 71 to enclose the absorbent middle layer, as described above. Posterior shield 32 and soft fabric layer 34 are disposed over the posterior portion 68 as described above, and adhesive strips disposed on the bottom layer of incontinence napkin 60 are used to attach the incontinence napkin 60 and the lateral edges of posterior portion 68 to the undergarment in the same manner as incontinence napkin 20, described above.

It is to be understood that the present invention is not limited to the embodiments described above, but encompasses any and all embodiments within the scope of the following claims.

1. A disposable incontinence napkin for men, comprising:
an elongated sanitary napkin having a liquid permeable top layer, a liquid impermeable bottom layer, and an absorbent middle layer disposed between the top layer and the bottom layer, the sanitary napkin having an anterior portion, a posterior portion, and a medial portion, the sanitary napkin being adapted for attachment to an undergarment with the anterior portion opposite a man's upper pubic area and base of the penis and the posterior portion opposite the scrotum, the top layer facing the pubic area;
a posterior shield disposed over the posterior portion of the top layer, the posterior shield being made from a liquid impermeable material, whereby the scrotum is kept dry and protected from moisture in the top layer when the sanitary napkin absorbs urine discharged from the penis; and
a soft fabric layer disposed over the posterior shield in order to prevent chaffing of the scrotum by the posterior shield.

2. (canceled)

3. The disposable incontinence napkin according to claim 1, further comprising a pair of adhesive strips disposed laterally on the posterior portion of the bottom layer, the adhesive strips being adapted for attaching lateral edges of the posterior portion of said sanitary napkin to an undergarment in order to prevent bunching of the sanitary napkin around the scrotum.

4. The disposable incontinence napkin according to claim 1, further comprising a central strip of adhesive disposed on the bottom layer extending from the anterior portion to the posterior portion of said sanitary napkin, the central strip of adhesive being adapted for attaching said sanitary napkin to an undergarment opposite the pubic area.

5. The disposable incontinence napkin according to claim 1, wherein said middle layer comprises a loose agglomeration of absorbent fibers.

6. The disposable incontinence napkin according to claim 5, wherein said middle layer further comprises an elongated, absorbent pad disposed between said top layer and said loose agglomeration of fibers, the absorbent pad maintaining said sanitary napkin's shape and distributing absorbed liquids to said loose agglomeration of fibers.

7. A disposable incontinence napkin for men, comprising:
an elongated sanitary napkin having a liquid permeable top layer, a liquid impermeable bottom layer, and an absorbent middle layer disposed between the top layer and the bottom layer, the sanitary napkin having an anterior portion, a posterior portion, and a medial portion, the sanitary napkin being adapted for attachment to an undergarment with the anterior portion opposite a man's upper pubic area and base of the penis and the posterior portion opposite the scrotum, the top layer facing the pubic area; and
a pair of adhesive strips disposed laterally on the posterior portion of the bottom layer, the adhesive strips being adapted for attaching lateral edges of the posterior portion of said sanitary napkin to an undergarment in order to prevent bunching of the sanitary napkin around the scrotum.

8. The disposable incontinence napkin according to claim 7, further comprising a posterior shield disposed over the posterior portion of the top layer, the posterior shield being made from a liquid impermeable material, whereby the scrotum is kept dry and protected from moisture in the top layer when the sanitary napkin absorbs urine discharged from the penis.

9. The disposable incontinence napkin according to claim 8, further comprising a soft fabric layer disposed over said posterior shield in order to prevent chaffing of the scrotum by said posterior shield.

10. The disposable incontinence napkin according to claim 7, further comprising a central strip of adhesive disposed on the bottom layer extending from the anterior portion to the posterior portion of said sanitary napkin, the central strip of adhesive being adapted for attaching said sanitary napkin to an undergarment opposite the pubic area.

11. The disposable incontinence napkin according to claim 7, wherein said middle layer comprises a loose agglomeration of absorbent fibers.

12. The disposable incontinence napkin according to claim 11, wherein said middle layer further comprises an elongated, absorbent pad disposed between said top layer and said loose agglomeration of fibers, the absorbent pad maintaining said sanitary napkin's shape and distributing absorbed liquids to said loose agglomeration of fibers.

13. A disposable incontinence napkin for men, comprising:
a elongated sanitary napkin having a liquid permeable top layer, a liquid impermeable bottom layer, and an absorbent middle layer disposed between the top layer and the bottom layer, the sanitary napkin having an anterior portion, a posterior portion, and a medial portion, the sanitary napkin being adapted for attachment to an undergarment with the anterior portion opposite a man's upper pubic area and base of the penis and the posterior portion opposite the scrotum, the top layer facing the pubic area;
means for shielding the scrotum from moisture absorbed by the sanitary napkin in order to keep the scrotum dry; and
means for preventing the sanitary napkin from bunching around the scrotum.

14. The disposable incontinence napkin according to claim 13, wherein said means for shielding comprises a posterior shield disposed over the posterior portion of the top layer, the posterior shield being made from a liquid impermeable material, whereby the scrotum is kept dry and protected from moisture in the top layer when the sanitary napkin absorbs urine discharged from the penis.

15. The disposable incontinence napkin according to claim 14, further comprising a soft fabric layer disposed over said posterior shield in order to prevent chaffing of the scrotum by said posterior shield.

16. The disposable incontinence napkin according to claim 13, wherein said means for preventing the sanitary napkin from bunching comprises a pair of adhesive strips disposed laterally on the posterior portion of the bottom layer, the adhesive strips being adapted for attaching lateral edges of the posterior portion of said sanitary napkin to an undergarment in order to prevent bunching of the sanitary napkin around the scrotum.

17. The disposable incontinence napkin according to claim 13, further comprising a central strip of adhesive disposed on the bottom layer extending from the anterior portion to the posterior portion of said sanitary napkin, the central strip of adhesive being adapted for attaching said sanitary napkin to an undergarment opposite the pubic area.

18. The disposable incontinence napkin according to claim 13, wherein said middle layer comprises a loose agglomeration of absorbent fibers.

19. The disposable incontinence napkin according to claim 13, wherein said middle layer further comprises an elongated, absorbent pad disposed between said top layer and said loose agglomeration of fibers, the absorbent pad maintaining said sanitary napkin's shape and distributing absorbed liquids to said loose agglomeration of fibers.
